(19) United States
(12) Patent Application Publication
(10) Pub. No.: US 2018/0344680 A1
(43) Pub. Date: Dec. 6, 2018

(54) COMPOSITIONS AND METHODS FOR TREATMENT, AMELIORATION, AND PREVENTION OF DIABETES-RELATED SKIN ULCERS

(71) Applicant: THE ARIZONA BOARD OF REGENTS ON BEHALF OF THE UNIVERSITY OF ARIZONA, Tucson, AZ (US)

(72) Inventors: Donna D. Zhang, Tucson, AZ (US); Georg T. Wondrak, Tucson, AZ (US)

(21) Appl. No.: 16/060,742

(22) PCT Filed: Dec. 7, 2016
(86) PCT No.: PCT/US16/65315

§ 371 Date: Jun. 8, 2018

Related U.S. Application Data
(60) Provisional application No. 62/264,713, filed on Dec. 8, 2015.

(51) Int. Cl. A61K 31/26; A61K 31/11; A61P 17/02; A61K 31/522; A61K 31/4709; A61K 31/4365; A61K 31/235; A61K 38/18

(52) U.S. Cl. CPC A61K 31/26; A61K 31/11; A61P 17/02; A61K 38/1858; A61K 31/4709; A61K 31/4365; A61K 31/235; A61K 31/522

(57) ABSTRACT

Compositions and methods are provided for treating, ameliorating, and preventing diabetes-related skin ulcers in a mammalian subject comprising administering to the subject an effective amount of an NRF2 activating agent.

### CROSS-REFERENCE TO RELATED APPLICATIONS

This application claims priority to and the benefit of U.S. Provisional Application No. 62/264,713, filed Dec. 8, 2015, which is hereby incorporated by reference in its entirety.

### FIELD OF THE INVENTION

Compositions and methods are provided for treating, ameliorating, and preventing diabetes-related skin ulcers in a mammalian subject comprising administering to the subject an effective amount of an NRF2 activating agent.

### INTRODUCTION

In the U.S., diabetes has reached epidemic proportions. According to the American Diabetes Association, about 24 million people (8% of the total U.S. population) have diabetes, and nearly two million new cases are diagnosed in people aged 20 years or older each year. If current trends continue, 1 in 3 Americans will develop diabetes at some point in their lifetime, and those with diabetes will lose, on average, 10-15 years of life expectancy. Importantly, up to 25% of people with diabetes will develop a diabetic foot ulcer, resulting in 3 million diabetic foot ulcers annually in the U.S. alone. More than half of all foot ulcers will become infected, thus requiring hospitalization, and 1 in 5 will require an amputation that carries a high risk of mortality.

Without question, diabetes puts tremendous economic pressure on the U.S. healthcare system. Total costs (direct and indirect) of diabetes have reached $174 billion annually, and people with diagnosed diabetes have medical expenditures that are over two times higher than medical expenditures for people without diabetes. Hospitalization costs alone are $16,000 to $20,000 for a patient with a diabetic foot ulcer, and direct and indirect costs of an amputation range from $20,000 to $60,000 per patient. A recent study by researchers at the University of Chicago suggested that treatment costs for diabetes in the United States would reach $336 billion by the year 2034. Advanced, cost-effective treatment modalities for diabetes and its co-morbidities, including diabetic foot ulcers, are in great need, yet in short supply, globally. According to the American Diabetes Association, by the year 2025 the prevalence of diabetes is expected to rise by 72% to 324 million people worldwide.

Improved compositions and methods for treating diabetes-related skin ulcers are urgently needed.

### SUMMARY OF THE INVENTION

Experiments conducted during the course of developing embodiments for the present invention demonstrated the therapeutic implication of NRF2 activation in promoting diabetic wound healing. Using human clinical specimens, it was shown that perilesional skin tissues from diabetic patients are under more severe oxidative stress and display higher activation of the NRF2-mediated antioxidant response than perilesional skin tissues from normoglycemic patients. In an STZ-induced diabetes mouse model, Nrf2^{-/-} mice were shown to have delayed wound closure rates compared to Nrf2^{+/+} mice, which was, at least partially, shown to be due to greater oxidative DNA damage, low TGF-β1 and high MMP9 expression, and increased apoptosis. More importantly, pharmacological activation of the NRF2 pathway significantly was shown to improve wound healing. In vitro experiments in HaCaT cells confirmed that NRF2 contributes to wound healing by alleviating oxidative stress, increasing proliferation and migration, decreasing apoptosis, and increasing the expression of TGF-β1 and lowering MMP9 under hyperglycemic conditions.

Accordingly, in certain embodiments, the present invention provides methods for treating, ameliorating, and preventing a diabetes-related skin ulcer, comprising administering to a human patient suffering from a diabetes-related skin ulcer an effective amount of an NRF2 activating agent. In some embodiments, administration of the NRF2 activating agent reduces and/or prevents oxidative DNA damage within the diabetes-related skin ulcer. In some embodiments, administration of the NRF2 activating agent increases TGF-β1 expression within the diabetes-related skin ulcer. In some embodiments, administration of the NRF2 activating agent decreases MMP9 expression within the diabetes-related skin ulcer. In some embodiments, administration of the NRF2 activating agent reduces and/or prevents apoptosis within a diabetes-related skin ulcer.

Such methods are not limited to a particular type of NRF2 activating agent. In some embodiments, the NRF2 activating agent is able to activate NRF2 and/or NRF2 related pathways (e.g., NRF2/KEAP1 pathway) (see, e.g., Jung, et al., Molecules, 15:7266-7291, 2010). In some embodiment, the NRF2 activating agent is sulforaphane. In some embodiment, the NRF2 activating agent is cinnamaldehyde. In some embodiment, the NRF2 activating agent is bordoxolone methyl. In some embodiments, the NRF2 activating agent is a glutathione peroxidase-1 mimetic (e.g., ebselen). In some embodiments, the NRF2 activating agent is caffeic acid. In some embodiments, the NRF2 activating agent is resveratrol. In some embodiments, the NRF2 activating agent is curcumin. In some embodiments, the NRF2 activating agent is bixin. In some embodiments, the NRF2 activating agent is tanshinone I. In some embodiments, the NRF2 activating agent is tanshinone IIA. In some embodiments, the NRF2 activating agent is dihydrotanshinone. In some embodiments, the NRF2 activating agent is cryptotanshinone.

Such methods are not limited to a particular type of human patient. In some embodiments, the human patient is a human being suffering from diabetes mellitus. In some embodiments, the human patient is suffering from neuropathy. In some embodiments, the human patient is a human being at risk for developing a diabetes-related skin ulcer. In some embodiments, the human patient is a human being having a diabetes-related skin ulcer.

Such methods are not limited to a particular type of diabetes-related skin ulcer. In some embodiments, the diabetes-related ulcer is a chronic, non-healing skin ulcer. In some embodiments, the diabetes-related ulcer is a foot ulcer. In some embodiments, the foot ulcer is a neuropathy-related foot ulcer. In some embodiments, the foot ulcer is a trauma-related foot ulcer. In some embodiments, the foot ulcer is a deformity-related foot ulcer. In some embodiments, the foot ulcer is a high plantar pressure-related foot ulcer. In some embodiments, the foot ulcer is a callus formation-related foot ulcer. In some embodiments, the foot ulcer is an edema-related foot ulcer. In some embodiments, the foot ulcer is a peripheral arterial disease-related foot ulcer.

Such methods are not limited to a particular administration route. In some embodiments, the NRF2 activating agent is administered topically, orally, and/or intravenously. In some embodiments, the NRF2 activation agent is administered in a form selected from the group consisting of a cream form, a spray form, a dressing form, a patch form, a tablet form, and an intravenous form.

In some embodiments, the NRF2 activating agent is co-administered with one or more additional therapeutic agents effective for treating, ameliorating, and preventing diabetes-related skin ulcers. In some embodiments, the one or more additional therapeutic agents are selected from a hemorrheologic agent (e.g., pentoxifylline, cilostazol), an antiplatelet agent (e.g., clopidogrel, aspirin), and a wound healing agent (e.g., becaplermin).

In certain embodiments, the present invention provides methods for reducing and/or preventing oxidative DNA damage within a diabetes-related skin ulcer, comprising administering to a patient having a diabetes-related skin ulcer an NRF2 activating agent, wherein administration of the NRF2 activating agent results in a reduction and/or prevention of oxidative DNA damage within the diabetes-related skin ulcer.

In certain embodiments, the present invention provides methods for increasing TGF-β1 expression within a diabetes-related skin ulcer, comprising administering to a patient having a diabetes-related skin ulcer an NRF2 activating agent, wherein administration of the NRF2 activating agent results in an increase in TGF-β1 expression within the diabetes-related skin ulcer.

In certain embodiments, the present invention provides methods for decreasing MMP9 expression within a diabetes-related skin ulcer, comprising administering to a patient having a diabetes-related skin ulcer an NRF2 activating agent, wherein administration of the NRF2 activating agent results in a decrease in MMP9 expression within the diabetes-related skin ulcer.

In certain embodiments, the present invention provides methods for reducing and/or preventing apoptosis within a diabetes-related skin ulcer, comprising administering to a patient having a diabetes-related skin ulcer an NRF2 activating agent, wherein administration of the NRF2 activating agent results in reduction and/or prevention of apoptosis within the diabetes-related skin ulcer.

In certain embodiments, pharmaceutical formulations of NRF2 activating agents are provided.

In certain embodiments, the present invention provides kits comprising one or more NRF2 activating agents (e.g., sulforaphane, cinnamaldehyde) and instructions for administering the NRF2 agents to a subject. In some embodiments, the kits further comprise one or more additional therapeutic agents effective for treating, ameliorating, and preventing diabetes-related skin ulcers. In some embodiments, the one or more additional therapeutic agents are selected from a hemorrheologic agent (e.g., pentoxifylline, cilostazol), an antiplatelet agent (e.g., clopidogrel, aspirin), and a wound healing agent (e.g., becaplermin).

### BRIEF DESCRIPTION OF DRAWINGS

FIG. 1: The perilesional skin tissues of diabetic patients are under severe oxidative damage with activation of the NRF2/mediated antioxidant response. (A-L) Hematoxylin and eosin (HE) staining, immunohistochemistry (IHC), and apoptosis analysis of human skin tissue specimens. Human skin tissue samples from 11 normoglycemic and 12 diabetic patients were fixed and paraffin-embedded; the tissue sections were subjected to HE staining (A,B) and IHC analysis (C-J) (magnification: 200×). Apoptotic cells in the tissue were detected by transferase-mediated dUTP nick-end labeling (TUNEL) assay (K,L) (magnification: 100×). Representative images from the perilesional skin tissues of wounds are shown. Scale bar, 100 μm.

FIG. 2: Deletion of NRF2 delays wound healing in an STZ-induced diabetic mouse model. (A) Time line for treatments, surgery and wound healing assessment. Nrf2^{+/+} and Nrf2^{-/-} mice at 8 weeks of age received STZ intraperitoneal injections in 5 consecutive days to induce diabetes. 4 weeks later, mice with FGL higher than or equal to 250 mg/dL were considered diabetic. Each diabetic Nrf2^{+/+} and Nrf2^{-/-} mice had two wounds made in their backs. Wounds were photographed five times at the indicated time points before the skin tissues were harvested at day 14. (B) Representative photographs of wounds of diabetic Nrf2^{+/+} and Nrf2^{-/-} mice. The day after the wound surgery is indicated at the top. (C) Wound closure. Wound area was quantified and presented as the percentage of wound that healed up to 14 days after wound surgery. Data are expressed as means±SEM (n=10-12), comparisons of diabetic Nrf2^{+/+} vs. Nrf2^{-/-} mice were done with a Student's t test. *P<0.05 compared to diabetic Nrf2^{-/-} mice. (D) IHC analysis of wound skin tissues harvested at day 14 with the indicated antibodies; apoptotic cells in the tissue were detected by TUNEL assay (magnification: 100×).

FIG. 3: NRF2 pathway is activated by SF and CA in skin tissues of STZ, mice. (A) Time line for treatments, surgery and wound healing assessment in Nrf2^{+/+} mice. Diabetic Nrf2^{+/+} mice were generated by STZ injections as described above; non/diabetic controls were injected with sodium citrate instead of STZ. Three weeks later, diabetic mice (FGL≥250 mg/dL) were randomly allocated into STZ or STZ+treatment (SF or CA) groups. All animals received compounds or corn oil every other day until the end of the experiment. Wound surgeries were done after one week of compound treatments; 2 weeks later, the wound skin tissues were harvested. Groups; non-diabetic control (Con); diabetic: untreated (STZ), SF-treated (STZ+SF), CA-treated (STZ+CA). (B) Relative body weight and (C) blood glucose concentration. Data were analyzed by ANOVA and Tukey post hoc test. Results are expressed as means±SEM (n=5-8), *P<0.05 compared to Con, ^{#}P<0.05 compared to STZ. (D) Immunoblots of NRF2, HO-1, AKR1C1, NQO1, and actin using mouse wound skin tissues (each lane contains wound skin tissue lysates from an individual mouse, n=3 per group). (E) IHC analysis of NRF2 and HO-1 using mouse wound skin tissues. Representative images from each group are shown (magnification: 100×).

FIG. 4: NRF2 activation accelerates wound closure in STZ mice. (A) Representative photographs of wounds of Nrf2^{+/+} mice in different groups. (B) Wound closure. Wound area was quantified and presented as the percentage of wound that healed up to 14 days after wound surgery. Data were analyzed by ANOVA and Tukey post hoc test, results are expressed as means±SEM (n=10-16). *P<0.05 compared to Con, ^{#}P<0.05 compared to STZ. (C) Pathological assessment and diameter of mouse wound skin tissues 14 days after wound surgery. A representative image from one mouse per group is shown, the borders of the wound are indicated by dotted lines (magnification: 40×).

FIG. 5: SF and CA modulate the expression of TGF-β1 and MMP9, alleviate oxidative DNA damage, and decrease apoptosis of skin tissues in STZ mice. (A) Immunoblots of TGF-β1, MMP9, and actin using mouse wound skin tissue lysates. (B) IHC analysis of wound skin tissues using the indicated antibodies, as well as apoptosis by TUNEL assay. A representative image from each group is shown (magnification: 100×). (C) Infrared imaging of wound skin tissues. Representative photographs of wounds at the indicated days post-surgery (left) and quantification of temperature changes (ΔT) between wound area and surrounding healthy regions in the different treatment groups (right) are shown. Data were analyzed by ANOVA and Tukey post hoc test, results are expressed as means±SEM (n=10-16). *P<0.05 compared to Con, ^{#}P<0.05 compared to STZ.

FIG. 6: SF and CA activate the NRF2 pathway, modulate the expression of MMP9 and TGF-β, and alleviate oxidative stress in human keratinocytes under hyperglycemic condition. (A,B) Immunoblots of NRF2, HO-1, AKR1C1, NQO1 and Actin. HaCaT cells were incubated in either low glucose (LG) or high glucose (HG) medium for two days. HG cells were treated with SF or CA (HG+SF, HG+CA) for 48 h (A) or were transfected with the indicated siRNA (HG+ConsiRNA, HG+NRF2siRNA) for 72 h (B). Cell lysates were subjected to immunoblot analysis. (C,D) Immunoblots of MMP9 and zymography of extracellular MMP9. HaCaT cells were incubated and treated as above, during the last 24 h HaCaT cells were incubated with one half volume of the medium without FBS. Cells were harvested and subjected to immunoblot analysis. The medium was subjected to zymography analysis for detection of MMP9 proteolytic activity. (E,F) Immunoassay of TGF-β1 secreted to the medium. HaCaT cells were treated as above and the medium was harvested to detect the extracellular TGF-β1 levels. The mean values were used and normalized to the LG or LG+ConsiRNA group, represented as bar graphs. Data were analyzed by ANOVA and Tukey post hoc test, results are expressed as means±SEM (n=4). ^{#}P<0.05 compared to HG or HG+ConsiRNA. (G,H) ROS detection and quantification. Similarly treated HaCaT cells were undergone DCF/flow cytometry analysis for ROS detection. The mean fluorescence values were used and normalized to the LG or LG+ConsiRNA group, represented as bar graphs. Data were analyzed by ANOVA and Tukey post hoc test, results are expressed as means±SEM (n=3), *P<0.05 compared to LG or LG+ConsiRNA, ^{#}P<0.05 compared to HG or HG+ConsiRNA.

FIG. 7: SF and CA promote keratinocyte migration by activating NRF2. (A,B) In vitro wound healing assay of keratinocytes. HaCaT Cells were incubated in either low glucose (LG) or high glucose (HG) medium for two days. HG cells were treated with SF or CA (HG+SF, HG+CA) for 24 h before removal of PDMS slab to generate gaps (A) or were transfected with the indicated siRNA (HG+ConsiRNA, HG+NRF2siRNA) for 48 h, followed by removal of PDMS slab (B). Cells were incubated with new medium without or with SF or CA everyday up to 72 h. Representative cell images from each group in the indicated time points after removal of PDMS slab are shown; the white dotted lines represent the wound boundary (left panel). Quantification of wound healing is shown (right panel). Data were analyzed by ANOVA and Tukey post hoc test, results are expressed as means±SEM (n=4). *P<0.05 compared to LG or LG+ConsiRNA group, ^{#}P<0.05 compared to HG or HG+ConsiRNA.

FIG. 8: NRF2 activation increases proliferation and decreases apoptosis of keratinocytes. (A,B) Cell growth index. Similarly treated (a) or siRNA-transfected (B) HaCaT cells were monitored for cell growth up to 72 h in real time. Data are expressed as means±SEM (n=3). (C,D) Ki67 immunofluorescence images (top) and quantification of fluorescence intensity (bottom graph). Similarly treated (c) or siRNA-transfected (D) HaCaT cells were subjected to immunofluorescence analysis with Ki67 antibodies. The relative Ki67 expression was quantified, analyzed by ANOVA and Tukey post hoc test, results are expressed as means±SEM (n=3). *P<0.05 compared to LG or LG+ConsiRNA, ^{#}P<0.05 compared to HG or HG+ConsiRNA. (E,F) In situ cell death assessment by TUNEL assay (top) and quantification (bottom graph). Similarly treated (E) or siRNA-transfected (F) HaCaT cells were subjected for TUNEL analysis. For the positive control, cells were treated with 20 μmol/L cisplatin for 24 h. Relative cell apoptosis was quantified, analyzed by ANOVA and Tukey post hoc and the results are expressed as means±SEM (n=3), *P<0.05 compared to LG or LG+ConsiRNA, ^{#}P<0.05 compared to HG or HG+ConsiRNA.

FIG. 9: NRF2 activation by SF or CA does not improve wound healing in non-diabetic mice. (A) Representative photographs of wounds of non-diabetic Nrf2^{+/+} mice treated with corn oil (Con), sulforaphane (SF), or cinnamaldehyde (CA). (B) Wound closure. Wound area was quantified and presented as the percentage of wound that healed up to 14 days after wound surgery. Data were analyzed by ANOVA and Tukey post hoc test, results are expressed as means±SEM (n=6-10). No statistically significant differences were observed.

### DETAILED DESCRIPTION OF THE INVENTION

Chronic non-healing skin ulcers are a major cause of disability and mortality in the diabetic population (see, e.g., Boulton A J, et al., Lancet 366:1719-1724, 2005; Margolis D J, et al., Diabetes Care 34:2363-2367, 2011). Among diabetic Medicare beneficiaries, approximately 8% have foot ulcers with a mortality rate of 11%, and around 1.8% have lower extremity amputations with a mortality rate of 22% (see, e.g., Margolis D J, et al., Prevalence of Diabetes, Diabetic Foot Ulcer, and Lower Extremity Amputation Among Medicare Beneficiaries, 2006 to 2008: Data Points #1. 2011; Margolis D J, et al., Incidence of Diabetic Foot Ulcer and Lower Extremity Amputation Among Medicare Beneficiaries, 2006 to 2008: Data Points #2. 2011).

Cutaneous wound healing is a complex process consisting of inflammation, proliferation, vascularization and tissue remodeling (see, e.g., Gurtner G C, et al., Nature 453:314-321, 2008). Although biological events after cutaneous injury cannot be separated completely due to their integration and overlap, the wound healing process generally consists of 4 stages: coagulation, inflammation, migration-proliferation, and remodeling (see, e.g., Falanga V, Lancet 366:1736-1743, 2005). Platelets, neutrophils, monocytes, and macrophages are the main cell types involved during coagulation and inflammation, which last from several hours to several days. During these stages the fibrin plug is formed and many growth factors including transforming growth factor (TGF) are released (see, e.g., Martin P, et al., Development 131:3021-3034, 2004). TGF-β1 plays a crucial role in the recruitment of inflammatory cells as well as in the synthesis and deposition of extracellular matrix (ECM) (see, e.g., Pakyari M, et al., Critical Role of Transforming Growth Factor Beta in Different Phases of Wound Healing. Adv Wound Care (New Rochelle) 2:215-224, 2013). The migration-proliferation and remodeling stages occur several weeks after wounding and are sustained up to several months after. During these stages many events occur, such as ECM deposition, angiogenesis, migration, proliferation, contraction and tissue remodeling, with the participation of keratinocytes, fibroblasts and endothelial cells as primary cell types. In these stages keratinocytes produce TGF-β1 (see, e.g., Amjad S B, et al., Wound Repair Regen 15:748-755, 2007) which regulates angiogenesis, granulation tissue formation, ECM remodeling (see, e.g., Okuda K, et al., J Oral Pathol Med 27:463-469, 1998), re-epithelialization (see, e.g., Tredget E B, et al., Wound Repair Regen 13:61-67, 2005), and proliferation of keratinocytes and fibroblasts (see, e.g., Bamberger C, et al., Am J Pathol 167:733-747, 2005). Proliferation of keratinocytes and fibroblasts is important in wound healing since cell migration alone is insufficient to close large and full-thickness wounds (see, e.g., Martin P, Science 276:75-81, 1997). Other key factors essential for wound healing are matrix metalloproteinases (MMPs) that regulate ECM degradation and facilitate keratinocytes migration (see, e.g., Ravanti L, et al., Int J Mol Med 6:391-407, 2000).

Normal wound healing is an acute process involving the aforementioned four stages; however, in diabetic wounds intrinsic pathophysiological abnormalities (reduced blood supply, angiogenesis, impaired wound contraction and matrix turnover) and extrinsic factors (infection and repeated trauma) lead to delayed and aberrant wound healing processes (see, e.g., Falanga V, Lancet 366:1736-1743, 2005; Arya A K, et al., World J Diabetes 5:756-762, 2014). Furthermore, many studies have identified that chronic oxidative stress associates with the progression of diabetic complications and impaired wound healing (see, e.g., Ceriello A, et al., Diabetes 61:2993-2997, 2012; Baynes J W, Diabetes 40:405-412, 1991; Kant V, et al., Int Immunopharmacol 20:322-330, 2014; Fadini G P, et al., Diabetes 59:2306-2314, 2010).

The redox-sensitive transcription factor NRF2 (nuclear factor-E2-related factor 2), the crucial regulator of the adaptive response to exogenous and endogenous oxidative stresses, regulates the expression of an array of genes through antioxidant response elements in their promoters to neutralize free radicals and accelerate the removal of toxic environmental insults (see, e.g., Kensler T W, et al., Annu Rev Pharmacol Toxicol 47:89-116, 2007; Jaramillo M C, et al., Genes Dev 27:2179-2191, 2013). Recently, additional novel biological functions of NRF2 have been confirmed, such as its participation in cell migration (see, e.g., Riahi R, et al., Integr Biol (Camb) 6:192-202, 2014), proliferation (see, e.g., Yang J J, et al., Cell Signal 26:2381-2389, 2014), apoptosis (see, e.g., Niture S K, et al., J Biol Chem 286:44542-44556, 2014), and differentiation (see, e.g., Jang J, et al., Stem Cells, 2014; Whitman S A, et al., Exp Cell Res 319:2673-2683, 2013). NRF2 has been regarded as an attractive druggable target for many human diseases including cancer (see, e.g., Jaramillo M C, et al., Genes Dev 27:2179-2191, 2013), neurodegenerative diseases (see, e.g., Johnson J A, et al., Ann N Y Acad Sci 1147:61-69, 2008), liver cirrhosis (see, e.g., Wu T, et al., Genes Dev 28:708-722, 2014), diabetes (see, e.g., Chartoumpekis D V, et al., Curr Diabetes Rev 9:137-145, 2013) and wound healing (see, e.g., Hayashi R, et al., Free Radic Biol Med 61C:333-342, 2013). A protective role of NRF2 has been established (see, e.g., Jiang T, et al., Diabetes 59:850-860, 2010) and the potential therapeutic effect of NRF2 activators, sulforaphane (SF) and cinnamaldehyde (CA) (see, e.g., Zheng H, et al., Diabetes 60:3055-3066, 2011) in a diabetic nephropathy animal model.

Experiments conducted during the course of developing embodiments for the present invention explored role of NRF2 and the therapeutic potential of NRF2 activators in diabetic wound healing. In human wound skin specimens, more severe oxidative stress, as well as activation of the NRF2 pathway and increased apoptosis were detected in the wound skin tissues of diabetic patients compared to the wound skin tissues of trauma patients. In a streptozotocin (STZ)-induced diabetes mouse model, Nrf2 knockout (Nrf2^{-/-}) mice showed impaired wound healing compared to Nrf2 wild-type (Nrf2^{+/+}) mice. Furthermore, activation of the NRF2 pathway by SF or CA accelerated wound closure of STZ diabetic mice. In vitro experiments in immortalized human keratinocytes indicated that the activation of NRF2 pathway by SF or CA displayed beneficial effects such as reducing oxidative stress, promoting keratinocyte proliferation and migration, and inhibiting apoptosis. Such experiments indicate that the NRF2 signaling pathway contributes to the wound healing processes, and further indicates that NRF2 activators can be used to treat skin ulcers in diabetic patients.

Accordingly, compositions and methods are provided for treating, ameliorating, and preventing diabetes-related skin ulcers in a mammalian subject comprising administering to the subject an effective amount of an NRF2 activating agent

In some embodiments, treating a diabetes-related skin ulcer includes one or more of (a) limiting the progression in size, area, and/or depth of the ulcer; (b) reducing size, area, and/or depth of the ulcer; (c) increasing rate of healing and/or reducing time to healing; (d) healing of the ulcer (e.g., 100% epithelialization with no drainage); and (e) decreased incidence of amputation or slowing in time to amputation.

Compositions and methods are provided for increasing the wound closure rate for a diabetes-related skin ulcer in a mammalian subject comprising administering to the subject an effective amount of an NRF2 activating agent.

Compositions and methods are provided for reducing and/or preventing oxidative DNA damage within a diabetes-related skin ulcer in a mammalian subject comprising administering to the subject an effective amount of an NRF2 activating agent.

Compositions and methods are provided for increasing TGF-β1 expression within a diabetes-related skin ulcer in a mammalian subject comprising administering to the subject an effective amount of an NRF2 activating agent.

Compositions and methods are provided for decreasing MMP9 expression within a diabetes-related skin ulcer in a mammalian subject comprising administering to the subject an effective amount of an NRF2 activating agent.

Compositions and methods are provided for reducing and/or preventing apoptosis within a diabetes-related skin ulcer in a mammalian subject comprising administering to the subject an effective amount of an NRF2 activating agent.

Such compositions and methods are not limited to particular types or kinds of NRF2 activating agents. In some embodiments, the NRF2 activating agent is any agent able to increase the wound closure rate for a diabetes-related skin ulcer. In some embodiments, the NRF2 activating agent is any agent able to reduce and/or prevent oxidative DNA damage within a diabetes-related skin ulcer. In some embodiments, the NRF2 activating agent is any agent able to increase TGF-β1 expression within a diabetes-related skin ulcer. In some embodiments, the NRF2 activating agent is any agent able to reduce MMP9 expression within a diabetes-related skin ulcer. In some embodiments, the NRF2 activating agent is any agent able to reduce and/or prevent apoptosis within a diabetes-related skin ulcer.

In some embodiments, the NRF2 activating agent is able to activate NRF2 and/or NRF2 related pathways (e.g., NRF2/KEAP1 pathway) (see, e.g., Jung, et al., Molecules, 15:7266-7291, 2010). In some embodiment, the NRF2 activating agent is sulforaphane. In some embodiment, the NRF2 activating agent is cinnamaldehyde. In some embodiment, the NRF2 activating agent is bordoxolone methyl. In some embodiments, the NRF2 activating agent is a glutathione peroxidase-1 mimetic (e.g., ebselen). In some embodiments, the NRF2 activating agent is caffeic acid. In some embodiments, the NRF2 activating agent is resveratrol. In some embodiments, the NRF2 activating agent is curcumin. In some embodiments, the NRF2 activating agent is bixin. In some embodiments, the NRF2 activating agent is tanshinone I. In some embodiments, the NRF2 activating agent is tanshinone IIA. In some embodiments, the NRF2 activating agent is dihydrotanshinone. In some embodiments, the NRF2 activating agent is cryptotanshinone.

Such compositions and methods are not limited to a particular type of subject. In some embodiments, the subject is a human being suffering from diabetes mellitus. In some embodiments, the subject is a human being at risk for developing a diabetes-related skin ulcer. In some embodiments, the subject is a human being having a diabetes-related skin ulcer.

Such compositions and methods are not limited to a particular type or kind of a diabetes-related skin ulcer. In some embodiments, the diabetes-related skin ulcer is a chronic, non-healing skin ulcer.

In some embodiments, the diabetes-related skin ulcer is characterized by having a delayed wound closure rate in comparison with a wound closure rate of a non-diabetes-related skin ulcer. In some embodiments, the diabetes-related skin ulcer is characterized by oxidative DNA damage within the ulcer. In some embodiments, the diabetes-related skin ulcer is characterized by decreased TGF-β1 expression within the ulcer. In some embodiments, the diabetes-related skin ulcer is characterized by increased MMP9 expression within the ulcer. In some embodiments, the diabetes-related skin ulcer is characterized by increased apoptosis within the ulcer. In some embodiments, the diabetes-related skin ulcer is a diabetic foot ulcer. In some embodiments, the diabetes-related skin ulcer is a diabetic leg ulcer.

In some embodiments, the diabetes-related skin ulcer is a foot ulcer. In some embodiments, the diabetic foot ulcer is one caused, at least in part, by neuropathy, trauma, deformity, high plantar pressures, callus formation, edema, and peripheral arterial disease. In some embodiments, the human diabetic foot ulcer is one caused, at least in part, by neuropathy and resulting pressure (weight bearing on the extremity due to lack of feeling in the foot). As is known to those of skill in the art, human diabetic foot ulcers tend to be due to neuropathy and pressure, which differs significantly from, for example, murine acute wounds. In a further preferred embodiment, the diabetic foot ulcer comprises one or more calluses.\

In some embodiments, the NRF2 activating agent is co-administered with one or more additional therapeutic agents effective for treating, ameliorating, and preventing diabetes-related skin ulcers. In some embodiments, the additional therapeutic agent is a hemorrheologic agent (e.g., pentoxifylline, cilostazol). In some embodiments, the additional therapeutic agent is an antiplatelet agent (e.g., clopidogrel, aspirin). In some embodiments, the additional therapeutic agent is a wound healing agent (e.g., becaplermin).

The invention also provides pharmaceutical compositions comprising NRF2 activating agents in a pharmaceutically acceptable carrier.

The invention also provides kits comprising NRF2 activating agents and instructions for administering the agents to a subject. The kits may optionally contain other therapeutic agents, e.g., additional agents for treating diabetes-related skin ulcers.

Compositions within the scope of this invention include all compositions wherein NRF2 activating agents are contained in an amount which is effective to achieve its intended purpose (e.g., treating diabetic-related ulcers). While individual needs vary, determination of optimal ranges of effective amounts of each component is within the skill of the art. Typically, the NRF2 activating agents may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders (e.g., diabetic-related ulcers). For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the NRF2 activating agent. The unit dose may be administered one or more times daily as one or more tablets or capsules each containing from about 0.1 to about 10 mg, conveniently about 0.25 to 50 mg of the NRF2 activating agent or its solvates.

In a topical formulation, the NRF2 activating agent may be present at a concentration of about 0.01 to 100 mg per gram of carrier. In a one embodiment, the compound is present at a concentration of about 0.07-1.0 mg/ml, for example, about 0.1-0.5 mg/ml, and in one embodiment, about 0.4 mg/ml.

In addition to administering the NRF2 activating agent as a raw chemical, the NRF2 activating agents may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of the NRF2 activating agents, together with the excipient.

The pharmaceutical compositions of the invention may be administered to any patient which may experience the beneficial effects of the NRF2 activating agents. Foremost among such patients are mammals, e.g., humans, although the invention is not intended to be so limited. Other patients include veterinary animals (cows, sheep, pigs, horses, dogs, cats and the like).

The NRF2 activating agents and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a mariner which is itself known, for example, by means of conventional mixing, granulating, dragee-making, dissolving, or lyophilizing processes. Thus, pharmaceutical preparations for oral use can be obtained by combining the NRF2 activating agents with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of doses.

Other pharmaceutical preparations which can be used orally include push-fit capsules made of gelatin, as well as soft, sealed capsules made of gelatin and a plasticizer such as glycerol or sorbitol. The push-fit capsules can contain the NRF2 activating agents in the form of granules which may be mixed with fillers such as lactose, binders such as starches, and/or lubricants such as talc or magnesium stearate and, optionally, stabilizers. In soft capsules, the NRF2 activating agents are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the NRF2 activating agents with a suppository base. Suitable suppository bases are, for example, natural or synthetic triglycerides, or paraffin hydrocarbons. In addition, it is also possible to use gelatin rectal capsules which consist of a combination of the NRF2 activating agents with a base. Possible base materials include, for example, liquid triglycerides, polyethylene glycols, or paraffin hydrocarbons.

Suitable formulations for parenteral administration include aqueous solutions of the NRF2 activating agents in water-soluble form, for example, water-soluble salts and alkaline solutions. In addition, suspensions of the NRF2 activating agents as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of NRF2 activating agents are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762; each herein incorporated by reference in its entirety.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

The examples described in Examples I-IX demonstrate a protective role for NRF2 against impaired diabetic wound healing. First, perilesional tissues from diabetic and normoglycemic patients were analyzed and the results indicated that the diabetic wound skin tissues were under more severe oxidative stress than normal wound skin tissues, as demonstrated by greater oxidative DNA damage and activation of the NRF2 pathway. Next, Nrf2^{+/+} and Nrf2^{-/-} mice were used to demonstrate the importance of NRF2 in improving the impaired wound healing process in STZ-induced diabetes model. Diabetic Nrf2^{-/-} mice showed delayed wound closure when compared to diabetic Nrf2^{+/+} mice, which may be due to greater oxidative DNA damage, altered TGF-β1 and MMP9 expression, and increased apoptosis. Then, the therapeutic potential of NRF2 activators to restore normal wound healing was demonstrated in the Nrf2^{+/+} STZ-induced diabetic mouse model. In vitro experiments with HaCaT human keratinocytes further confirmed that NRF2 contributes to important events of wound healing including oxidative stress attenuation, promotion of proliferation and migration, and decreased apoptosis under high glucose incubation. These beneficial effects are, at least partially, through modulation of the expression of TGF-β1 and MMP9.

### Example 1

This example provides the materials and methods for Examples I-IX.

Chemicals, antibodies and cell culture. Cinnamaldehyde (CA), streptozotocin (STZ), and 2′,7′-dichlorofluorescein diacetate (DCF) were purchased from Sigma (St. Louis, Mo.). L-sulforaphane (SF) was obtained from LKT laboratories (St. Paul, Minn.). Primary antibodies against Ki67, NRF2, MMP9, HO-1, AKR1C1, TGF-β1, and Actin, as well as horseradish peroxidase (HRP)-conjugated secondary antibodies were from Santa Cruz Biotechnology, CA. Anti-8-dihydro-2-deoxyguanosine (8-oxo-dG) antibody was purchased from Trevigen. Inc. (Gaithersburg, Md.).

Human immortalized keratinocytes HaCaT were obtained from the Arizona Cancer Center (see, e.g., Boukamp P, et al., J Cell Biol 106:761-771, 1988). Cells were grown in Dulbecco's modified Eagle's medium (DMEM) containing low glucose (LG, 5.5 mmol/L) and 10% fetal bovine serum (FBS) in an incubator at 37° C. with 5% CO₂. For the experiments, the cells were starved in serum-free LG DMEM for 24 h, then either maintained in LG DMEM or switched to high glucose (HG, 25 mmol/L) DMEM for 2 days (see, e.g., Zheng H, et al., Diabetes 60:3055-3066, 2011; Lan C C, et al., Br J Dermatol 159:1103-1115, 2008). For the treatments, the cells were dosed with 5 μmol/L SF or 20 μmol/L CA.

siRNA transfections. HaCaT cells were transfected with either a non-specific, control small interfering RNA (Con-siRNA, #1027281, Qiagen, Valencia, Calif.) or an NRF2-specific siRNA (#S100657937) using the HiPerfect transfection reagent (Qiagen) according to the manufacturer's instructions. Briefly, cells were maintained in LG or HG DMEM for 2 days and then transfected with either control or NRF2 siRNA for migration, proliferation, and apoptosis experiments. For the ROS detection, cells were maintained in LG DMEM and transfected with either control or NRF2 siRNA; 24 h later, the cells were switched to HG DMEM and treated as indicated.

Human skin tissue samples. Diabetic skin tissue samples were obtained from ulcers of 12 patients with diabetes mellitus; normoglycemic skin samples were obtained from 11 patients who needed debridement due to trauma and did not suffer from serious diseases, such as diabetes, general infection, and cardiovascular or renal diseases. All tissue samples included a 1 cm margin surrounding the wound.

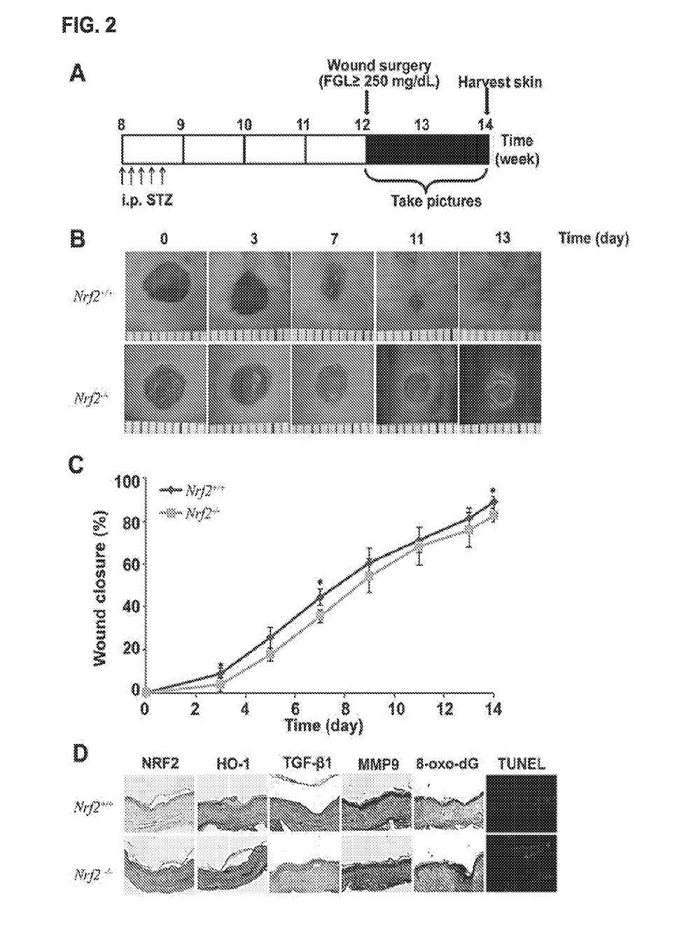

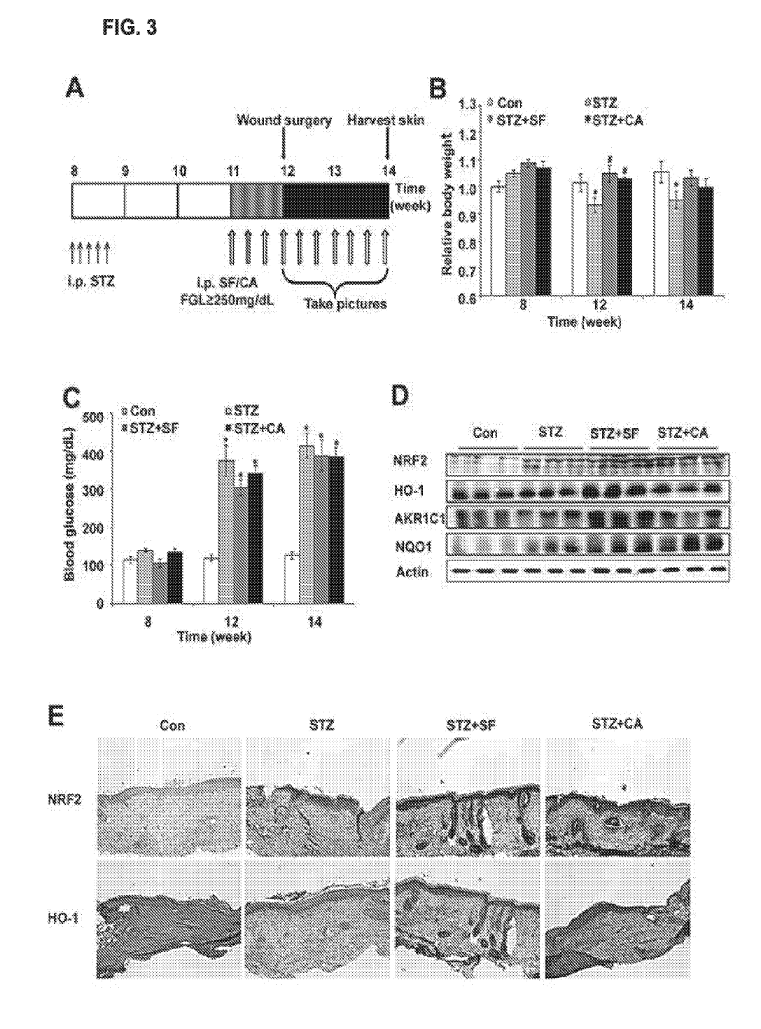

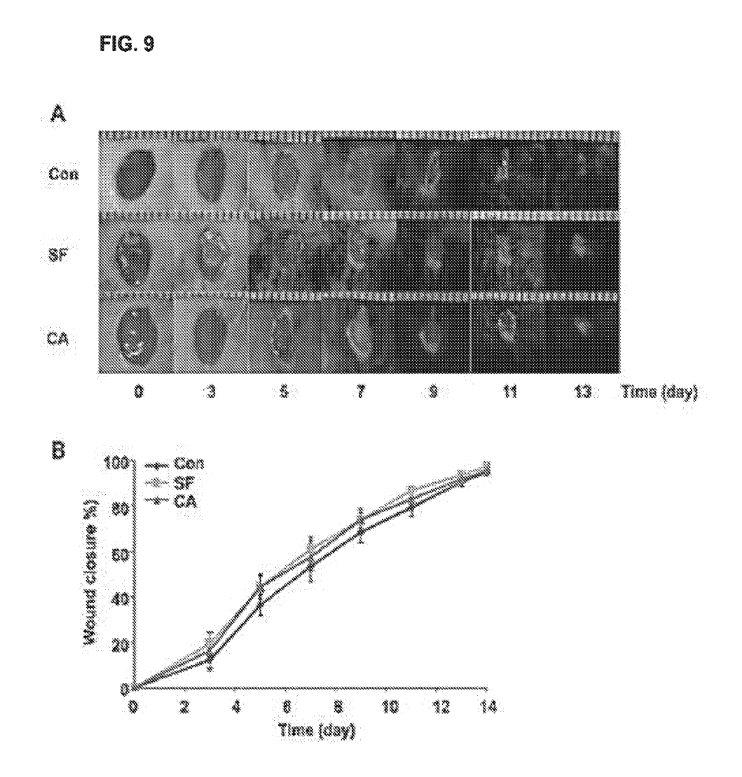

Diabetes mouse model and treatments. Nrf2^{+/+} and Nrf2^{-/-} C57BL/6 mice were described previously (see, e.g., Jiang T, et al., Diabetes 59:850-860, 2010) and were housed and handled in accordance with the University of Arizona Institutional Animal Care policies. The STZ-induced diabetic model was previously described (see, e.g., Zheng H, et al., Diabetes 60:3055-3066, 2011) and only 8 week old male mice were included in this study. Briefly, three weeks after STZ injections, fasting glucose levels (FGL, 4 h fast) were measured using the AlphaTRAK system (Abbott Laboratory, North Chicago, Ill.) and mice with a FGL higher than or equal to 250 mg/dL were considered diabetic and included in the study (see, e.g., FIG. 2A and FIG. 3A for a detailed timeline). The mice (n=5-8) were then allocated randomly to each of the indicated treatment groups and received corn oil (control), 12.5 mg/kg SF or 50 mg/kg CA intraperitoneally every two days until skin tissues were harvested. One week after the treatments, the mice were anesthetized, their backs were shaved and cleaned. Two wounds were made with a sterile 6 mm skin biopsy punch (Health link, Jacksonville, Fla.) and covered with 3M Tegaderm pads. The wounds were photographed using an in vivo imaging system and an infrared integrated system (IRISYS, Northampton, UK) for 14 days. Gross wound closure was quantified by ImageJ software, and wound healing was expressed as the percentage of the original wound area that had healed. The tissues were collected using an 8 mm skin biopsy punch; one half was fixed in 10% buffered formalin and embedded in paraffin while the other half was used for protein extraction. For the experiments in non-diabetic mice (FIG. 9), mice were injected with sodium citrate buffer and randomly allocated to the treatment groups (n=3-5). Wounds and tissue collection were performed as described above.

Immunohistochemistry (IHC), immunoblot analyses, TGF-β1 immunoassay, and gelatin zymography. Human and mouse skin tissue paraffin sections (3 μm) were baked and deparaffinized. Morphology was assessed by hematoxylin and eosin (H&E) staining (Vector laboratory, Burlingame, Calif.). For IHC, antigen retrieval was performed by boiling in sodium citrate buffer (10 mM, pH 6.0) or EDTA buffer (1 mM, pH 8.0). After blocking endogenous peroxidase and non-specific binding, tissue sections were incubated with the indicated primary antibodies. Staining was performed using the Envision system HRP-DAB kit (DAKO North America, CA) according to the manufacturer's instructions.

For protein detection of mouse skin tissues were homogenized in lysis buffer (50 mM Tris-HCl buffer pH 7.4, 100 mM DTT, 2% SDS, 10% glycerol). Protein concentration was measured using Quant-iT Protein Assay (Invitrogen) and 150 μg of protein were resolved by SDS-PAGE and immunoblotting analyses with the indicated antibodies.

Cultured HaCaT cells were harvested in 1× sample buffer (50 mM Tris-HCl pH 6.8, 2% sodium dodecyl sulfate (SDS), 10% glycerol, 100 mM dithiothreitol (DTT), 0.1% bromophenol blue), boiled and sonicated. Total cell lysates were resolved by SDS-PAGE and subjected to immunoblot analyses with the indicated antibodies. For the detection of the secreted proteins TGF-β1 and MMP9, cells were incubated 24 h in serum-free media before harvesting the medium. TGF-β1 was measured using the Quantikine human TGF-β1 immunoassay kit (R&D systems; Minneapolis, Minn.) according to the manufacturer's instructions. The absorbance at 450 nm was measured using a Synergy 2 microplate reader (BioTek, Winooski, Vt.) and the amount of TGF-β1 was calculated by standard curve. MMP9 activity was evaluated by gelatin zymography as described elsewhere (see, e.g., Toth M, et al., Methods Mol Biol 878:121-135, 2012; Zhu P, et al., Exp Dermatol 21:123-129, 2012). Briefly, the supernatant was concentrated using Amicon Ultra-0.5 mL centrifugal filters (Merck Milipore, Darmstadt, Germany) (10 kDa cut off), mixed with non-reducing sample buffer and subjected to electrophoresis in 10% polyacrylamide-0.1% w/v gelatin gels. The gels were then washed in 2.5% Triton X-100 to remove SDS and incubated with developing buffer (50 mM Tris-HCl, pH 7.6, 1 μM ZnCl₂ and 5 mM CaCl₂) at 37° C. for 36 h. Gels were subsequently stained with 0.2% Coomassie blue and were analyzed with ImageJ.

In vitro wounding assay. To create an empty space for cell migration with minimal injury to the cells in the border, a polydimethylsiloxane (PDMS) barrier for wound healing assays was utilized as described elsewhere (see, e.g., Anon E, et al., Proc Natl Acad Sci USA 109:10891-10896, 2012; Poujade M, et al., Proc Natl Acad Sci USA 104:15988-15993, 2007). Blocks of UV-sterilized PDMS (1 mm×2 cm) were placed transversally onto 35 mm glass bottom dishes, making sure the bottom of the block and the dish made contact completely to prevent cells from entering the space under the slab. After a confluent cell monolayer was formed, the slab was removed carefully to minimize the mechanical stress on the cell boundary and the cells were allowed to migrate for 72 h (see, e.g., Riahi R, et al., Integr Biol (Camb) 6:192-202, 2014). The cells were treated with SF or CA when seeding them and retreated every 24 h until the end of this experiment. For siRNA experiments, the cells were seeded at a density of 100,000 cells/mL. A confluent cell monolayer was formed after 48 h and the slab was removed to allow migration for 72 h.

Reactive oxygen species (ROS) measurement, cell proliferation and apoptosis. ROS were measured in HaCaT cells using DCF. Briefly, the cells were seeded in LG or HG medium and treated with SF or CA every 24 h. Then, 48 h later, the cells were switched into fresh medium containing 10 μg/mL DCF, incubated 30 min, and fluorescence intensity was measured by flow cytometry. The rate of cell proliferation was measured by two methods (see, e.g., Ren D, et al., Proc Natl Acad Sci USA 108:1433-1438, 2011): 1) detection of Ki67 using indirect immunofluorescence as described previously (see, e.g., Lau A, et al., Mol Cell Biol 30:3275-3285, 2010); and 2) using the xCELLigence system (Roche). For this, 8000 HaCaT cells/well were seeded in LG or HG media, with or without an NRF2 activator, and cell growth was monitored for 72 h. The In Situ Cell Death Detection Kit (Roche, Mannheim, Germany) was used to detect apoptosis according to the manufacturer's instructions. Fluorescent images were taken using a Zeiss Observer.Z1 microscope with the Slidebook 5.0 software (Intelligent Imaging Innovations, Denver, Colo.).

Statistical analysis. Results are expressed as means standard error mean (SEM). Statistical tests were performed using GraphPad Prism software (version 6.0). In vitro experiments were done in triplicate. One-way ANOVA with Tukey's post hoc test was applied to compare the means of three or more groups. Unpaired, two-sided Student's t tests were used to compare the means of two groups. P<0.05 was considered to be significant.

### Example II

This example describes that perilesional skin tissues of diabetic patients are under severe oxidative damage with activation of the NRF2-mediated antioxidant response.

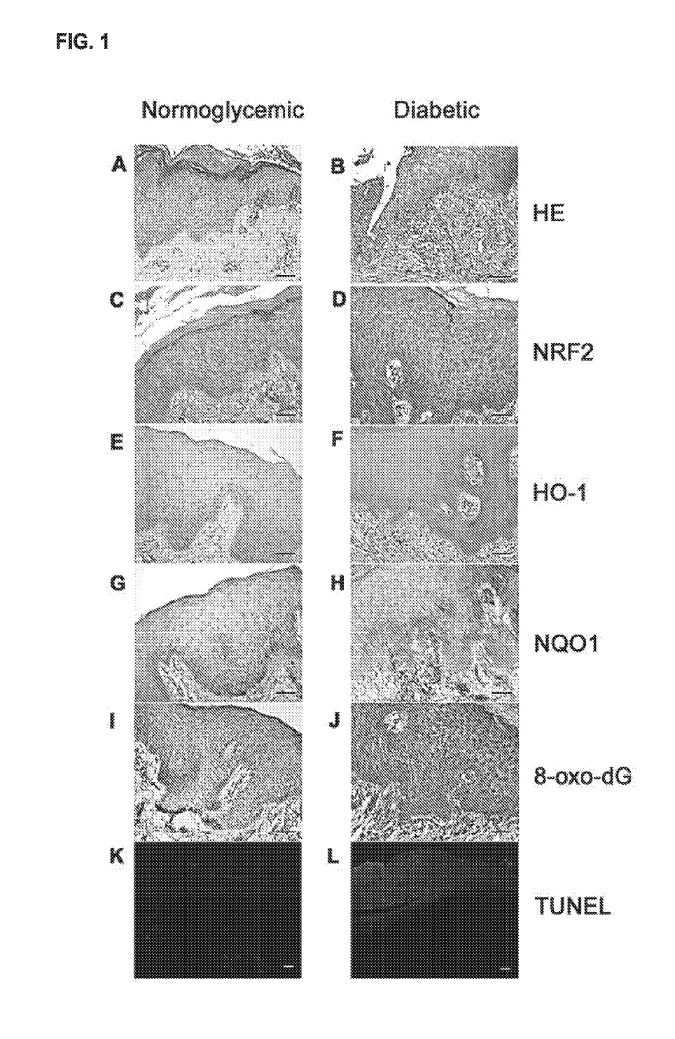

The perilesional skin tissues were collected from 11 normoglycemic and 12 diabetic patients for pathological (H&E) and immunohistochemical analyses (IHC). Compared to the normoglycemically perilesional skin tissue, the diabetically perilesional tissue has more inflammatory cells infiltration, less granulation tissue formation, and edema, indicating an impaired healing process in hyperglycemic condition (FIG. 1A,B). IHC analyses showed that NRF2 and its downstream targets HO-1 and NQO1 were much more highly expressed in diabetic skin than in the skin of trauma patients (FIG. 1C-H). Moreover, NRF2 was mostly expressed in the epidermal layer. Next, oxidative DNA damage was measured using IHC with an anti-8-oxo-dG antibody. Only moderate staining was detected in the trauma skin tissue whereas a stronger staining of 8-oxo-dG was detected in diabetic skin, indicating that skin adjacent to wounds is wider oxidative stress and diabetic wound tissue has undergone a higher level of oxidative DNA damage (FIG. 1I,J). In addition, a marked induction of apoptosis was observed in the skin of diabetic patients, as identified by TUNEL, but was not present in that of trauma patients (FIG. 1K,L). Together, these results indicate that skin tissues of diabetic patients undergo severe oxidative damage with activation of the NRF2-mediated antioxidant response combined with increased levels of apoptosis.

### Example III

This example demonstrates that deletion of Nrf2 delays wound healing in an STZ-induced diabetic mouse model.

Since diabetic skin tissues from human patients display a high level of oxidative stress, the role of NRF2 in wound healing was further investigated in an STZ-induced diabetes mouse model using Nrf2^{+/+} and Nrf2^{-/-} C57BL/6 mice. Following induction of diabetes by five STZ injections, two wounds were made in the backs of diabetic Nrf2^{+/+} and Nrf2^{-/-} mice and were photographed to compare and quantify wound closure (FIG. 2A). A gross examination of the wounds revealed there was an obvious delay in wound healing of diabetic Nrf2^{-/-} mice compared to diabetic Nrf2^{+/+} mice (FIG. 2B). The wound closure of diabetic Nrf2^{-/-} mice was slower throughout the entire healing process (FIG. 2B,C). At day 14, the wound skin tissues were harvested to analyze the expression of proteins by IHC. As expected, diabetic Nrf2^{-/-} mouse skin had no detectable levels of NRF2, whereas the expression of NRF2 in diabetic Nrf2^{+/+} mouse skin was obvious (FIG. 2D). Activation of the NRF2 signaling pathway was confirmed in Nrf2^{+/+} mouse skin by detection of HO-1 expression while its expression was below the detection limit in Nrf2^{-/-} mouse skin (FIG. 2D). Moreover, diabetic Nrf2^{-/-} mouse skin had lower TGF-β1 and higher MMP9 expression than Nrf2^{+/+} mouse skin (FIG. 2D), suggesting that changes in the expression of TGF-β1 and MMP9 may contribute to the delayed wound healing observed in diabetic Nrf2^{-/-} mice. An increase in 8-oxo-dG expression was detected in diabetic Nrf2^{-/-} mouse skin compared to diabetic Nrf2^{+/+} mouse skin, indicating that the wound skin tissues of diabetic Nrf2^{-/-} mice undergo greater oxidative DNA damage (FIG. 2D). Furthermore, more cells underwent apoptosis in diabetic Nrf2^{-/-} than in diabetic Nrf2^{+/+} mouse skin tissues. All these results indicate that diabetic Nrf2^{-/-} mice suffered delayed wound healing due to lack of NRF2-mediated antioxidant response, higher oxidative stress, low expression of TGF-β1 and high MMP9, and increased apoptosis. These results suggest that NRF2 plays an essential role in promoting diabetic wound healing.

### Example IV

This example demonstrates that NRF2 pathway is activated by SF and CA in skin tissues of STZ mice.

To explore the feasibility of pharmacologic activation of NRF2 in facilitating diabetic wound healing, sulforaphane (SF) and cinnamaldehyde (CA), two well-studied NRF2 activators, were tested for their ability to activate the NRF2-mediated antioxidant response in mouse skin tissues. After induction of diabetes using STZ, diabetic Nrf2^{+/+} mice were intraperitoneally injected with corn oil (vehicle control), 12.5 mg/kg SF, or 50 mg/kg CA every other day for 1 week, then the wound surgery was performed and the treatment continued for 2 more weeks (FIG. 3A). Untreated diabetic mice (STZ) had lower body weights (FIG. 3B) and higher blood glucose levels (FIG. 3C) than non-diabetic mice (Con), as expected. Treatment of diabetic mice with SF (STZ+SF) or CA (STZ+CA) reversed the body weight loss during the initial phase of diabetes (FIG. 3B) but failed to decrease blood glucose levels (FIG. 3C). Immunoblot analyses demonstrated that SF and CA upregulated the protein expression of NRF2 and its target genes HO-1, AKR1C1, and NQO1 in diabetic mouse skin tissue lysates (FIG. 3D). Furthermore, IHC data also confirmed the high level of NRF2 and HO-1 in wound skin tissues of diabetic mice treated with SF or CA (FIG. 3E). Taken together, these results indicate that SF and CA are able to activate the NRF2-mediated antioxidant response in mouse skin.

### Example V

This example demonstrates that NRF2 activation accelerates wound closure in STZ mice.

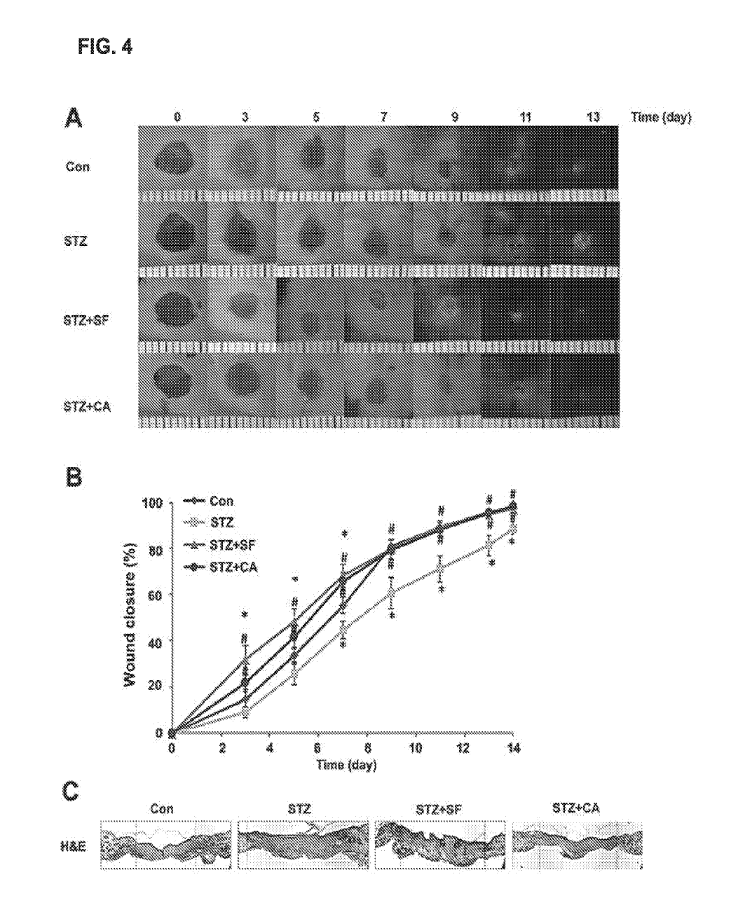

Since SF and CA can induce the NRF2-mediated antioxidant response in mouse skin, the effect of NRF2 activation in diabetic wound healing was further investigated. The wounds of diabetic mice (STZ) healed slower than the wounds of non-diabetic mice (Con), but treatment of STZ mice with either SF or CA accelerated wound closure (FIG. 4A,B). Interestingly, the wound closure of STZ+SF mice was even faster than that of the Con mice during the first 7 days (FIG. 4b). Histological examination of the skin tissues showed that the diameter of wounds at day 14 post-surgery in untreated STZ mice was the widest of all, while SF and CA markedly reduced the diameter of granulation tissue between the flanking hair follicles to resemble that of the Con mice (FIG. 4C). Nevertheless, non-diabetic mice treated with either SF or CA do not show accelerated wound closure (Supplementary Fig. S1). In summary, these results indicate that NRF2 activation by SF or CA promoted the wound healing process in STZ mice.

### Example VI

This example demonstrates that SF and CA modulate the expression of TGF-β1 and MMP9, alleviate oxidative DNA damage, and decrease apoptosis of skin tissues in STZ mice.

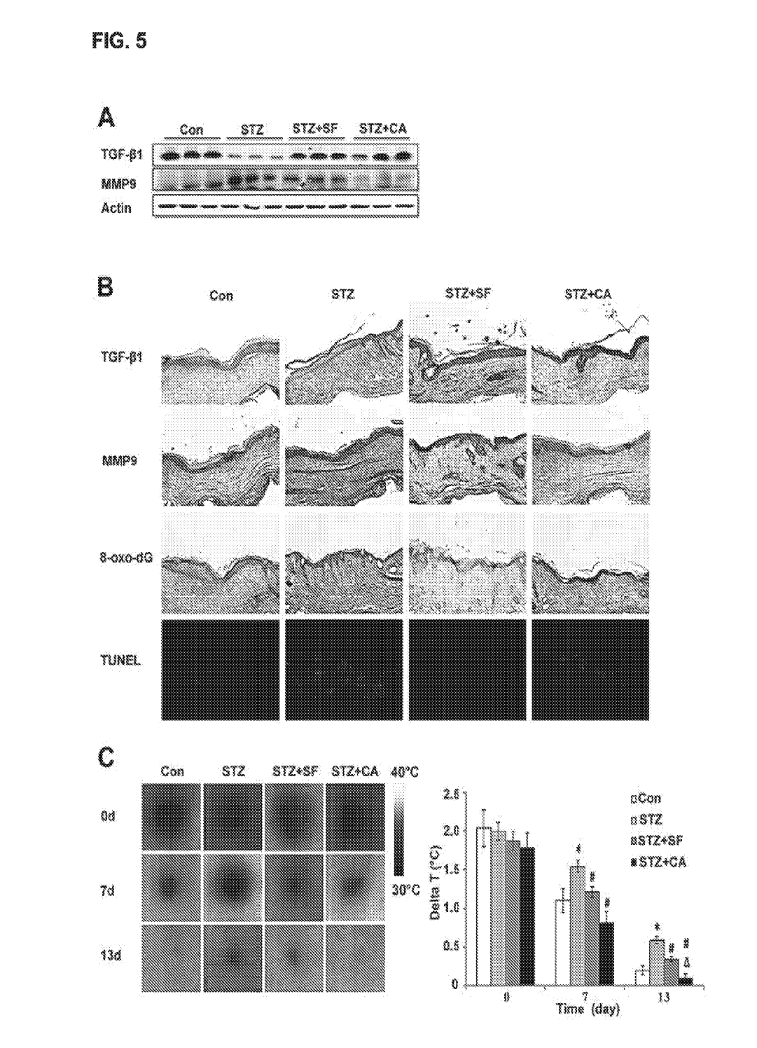

To investigate the molecular mechanisms by which SF and CA improve wound healing in the diabetic mouse model their effects on proliferation and apoptosis were studied. The expression of TGF-β1 and MMP9, which play crucial roles in the proliferative and remodeling phases during wound healing, was detected by immunoblot analyses and IHC. Wound skin tissues of STZ mice had lower TGF-β1 protein levels than Con mice, and STZ+SF or STZ+CA mice had TGF-β1 expression restored to levels comparable to that of Con mice (FIG. 5A,B). In contrast, STZ mice had the highest expression of MMP9 but treatment with SF or CA restored the protein expression of MMP9 to levels comparable to the Con mice. Therefore, the low TGF-β1 and high MMP9 protein expression in the STZ mice might partly explain their delayed wound healing. Next, oxidative DNA damage was also assessed by detection of 8-oxo-dG. Wound skin tissues from STZ mice had the highest expression of 8-oxo-dG, but treatment with SF or CA greatly alleviated the oxidative DNA damage (FIG. 5B). The extent of apoptosis induction in wound skin tissues was assessed by TUNEL. While wound skin tissues of STZ mice had a large extent of apoptosis, SF and CA treatments reduced apoptosis to a lesser extent than in Con (FIG. 5B).

Next, digital infrared thermal imaging was used to measure the difference in temperature between wound areas and the surrounding healthy tissue. As a noninvasive and high-resolution technique infrared thermography is widely used to assess hemodynamic and neurogenic variations in the tissues of patients with skin diseases (see, e.g., Otsuka K, et al., IEEE Eng Med Biol Mag 21:49-55, 2002) or diabetes mellitus (see, e.g., Armstrong D G, et al., Am J Med 120:1042-1046, 2007; Armstrong D G, et al., Phys Ther 77:169-175; discussion 176-167, 1997). Previous studies have identified that high temperature gradients may predict bad prognosis for diabetic patients with foot ulcers (see, e.g., Armstrong D G, et al., Am J Med 120:1042-1046, 2007). In this study, thermal imaging of the wounds showed that the temperature gradients in STZ mice were markedly exaggerated compared with the Con group; however, SF and CA abolished this effect (FIG. 5C). Taken together, these results indicate that SF and CA help promote diabetic wound healing by modulating the expression of TGF-β1 and MMP9, alleviating oxidative stress damage, and decreasing apoptosis of wound skin tissues in STZ mice.

### Example VII

This example demonstrates that SF and CA activate the NRF2 pathway, modulate the expression of MMP9 and TGF-β, and alleviate oxidative stress in human keratinocytes under hyperglycemic condition.

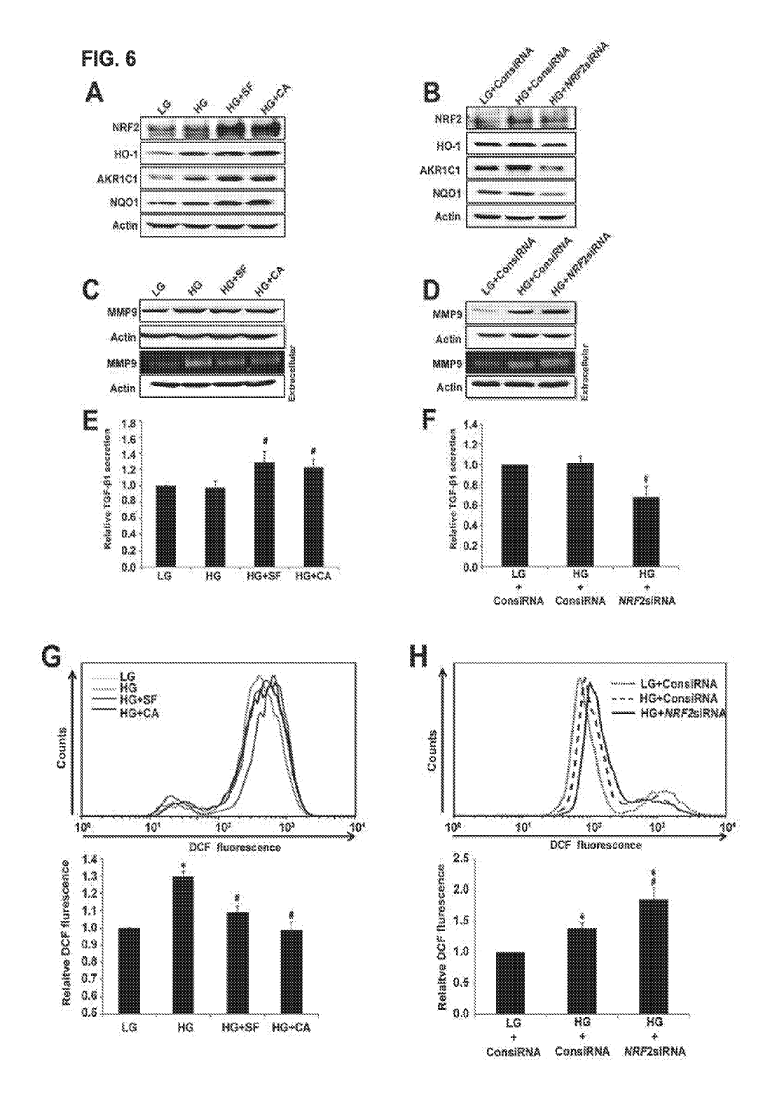

Keratinocytes play a crucial role in the wound healing process. In order to understand the molecular mechanisms of NRF2-dependent acceleration of diabetic wound healing cell based assays were performed using an immortalized human keratinocyte cell line (HaCaT). HaCaT cells were first cultured in low-glucose (LG) media for 2 weeks and then shifted to high-glucose (HG) media to mimic diabetic hyperglycemic conditions. Similar to the results reported above in the diabetic mouse model culturing HaCaT cells under hyperglycemic conditions activated the NRF2 pathway, as shown by an increase in the protein levels of NRF2 in HG cells after 48 h incubation and further NRF2 induction by SF or CA treatment (FIG. 6A). Similarly, protein levels of HO-1, AKR1C1, and NQO1 also increased in HG and were further induced by treatment with SF and CA (FIG. 6A). However, in HG+NRF2-siRNA cells induction of the NRF2 pathway decreased to a level comparable to that of LG+Con-siRNA cells (FIG. 6B).

Since the expression of MMP9 and TGF-β1 were altered in the epidermal layer of diabetic mouse skin tissues, their expression in HaCaT cells was also investigated. HG cells had high intracellular MMP9 as well as higher activity of the secreted MMP9 (FIG. 6C). Moreover, NRF2 negatively modulated the expression and activity of MMP9 in HG cells, as it decreased with SF or CA treatment but increased in HG+NRF2-siRNA (FIG. 6C,D). On the other hand, HG did not exhibit a significant increase in the secretion of TGF-β1 with respect to LG, but there seems to be a positive correlation between extracellular TGF-β1 secretion and NRF2 levels in HG cells (FIG. 6E,F).

Next, oxidative stress in HG cells was measured. Indeed, these cells had higher ROS levels than LG cells (FIG. 6G). Given the role of NRF2 in controlling the cellular response to oxidative stress, the effect of NRF2 upregulation or silencing in HG conditions was tested. As predicted, activation of NRF2 by SF or CA reduced ROS levels significantly (FIG. 6G), and knockdown of NRF2 further increased ROS levels in HG condition (FIG. 6H). All these results indicate that a hyperglycemic condition induces oxidative stress, activates the NRF2 pathway and alters the expression of MMP9 in keratinocytes. Furthermore, these results demonstrate the beneficial effects of NRF2 activation in alleviating oxidative stress, inducing TGF-β1, and decreasing MMP9 of keratinocytes under hyperglycemic conditions to aid in the diabetic wound healing.

### Example VIII

This example demonstrates that SF and CA promote keratinocytes migration by activating NRF2.

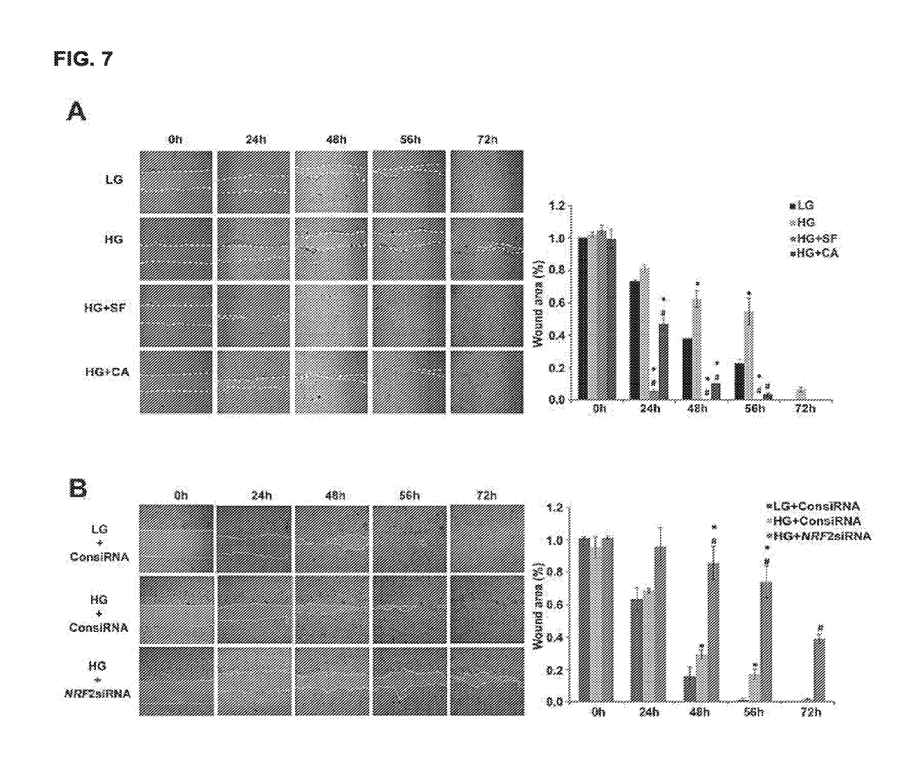

To further confirm the observation that the wound healing process was accelerated by NRF2 activation in the STZ-induced diabetic model, in vitro cell migration was studied. The PDMA-based wound healing assay was performed mimicking reepithelialization through keratinocyte migration. Indeed, cell migration was significantly inhibited in HG cells, which resulted in prolonged wound closure time (FIG. 7A). Nevertheless, SF and CA treatments significantly accelerated wound closure in HG cells (FIG. 7A), whereas HG+NRF2-siRNA cells had the slowest migration and the most delayed wound closure (FIG. 7B). Together, these results demonstrate that NRF2 is able to reverse impaired keratinocyte migration induced by HG.

### Example IX

This example demonstrates that NRF2 activation increases proliferation and decreases apoptosis of keratinocytes.

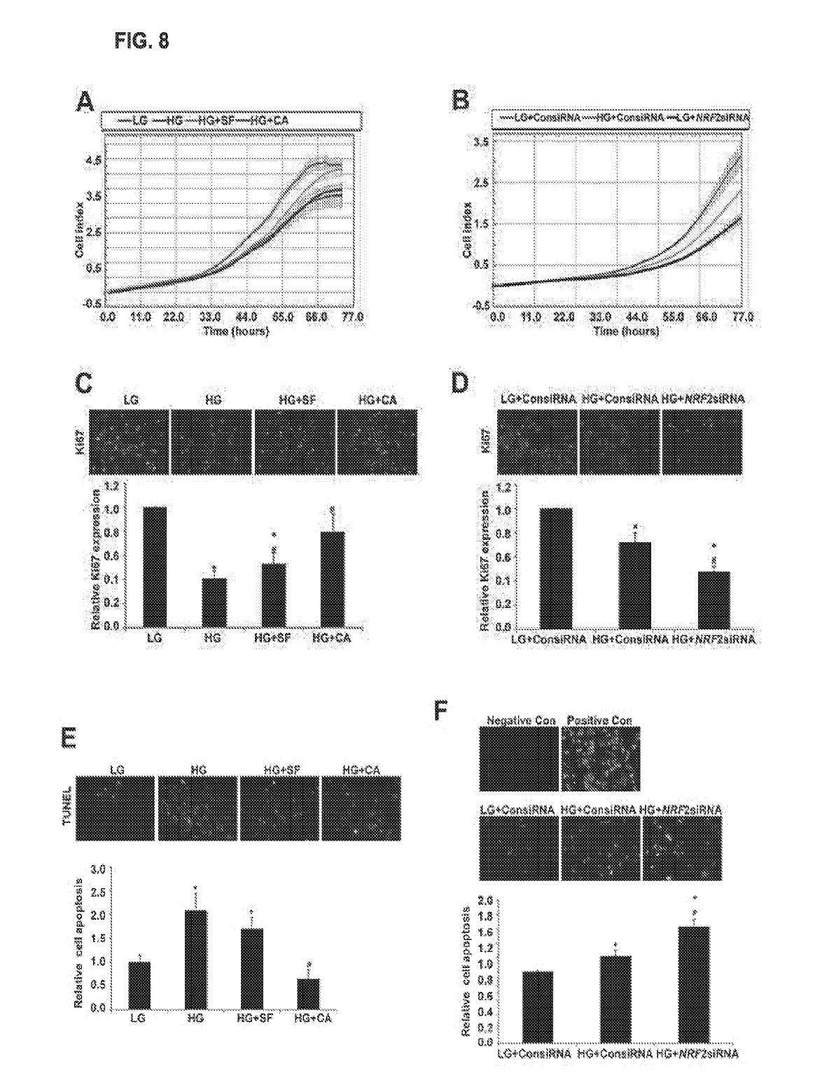

Since keratinocyte proliferation affects reepithelialization the growth rate of HaCaT cells was assessed. Cell proliferation was inhibited in HG cells which was partly counteracted by activation of NRF2 in HG+SF or HG+CA and exacerbated in HG+NRF2-siRNA cells (FIG. 8A,B). To further confirm this result, the cell proliferation marker Ki67 was detected by immunofluorescence. Accordingly, Ki67 expression in HG cells was lower than in LG, and treatment with SF or CA could restore some of the Ki67 expression (FIG. 8C). On the other hand, Ki67 expression further decreased in HG+NRF2-siRNA (FIG. 8D). These results demonstrate that NRF2 activation positively modulates keratinocyte proliferation. In contrast, increased levels of apoptosis were detected in HG cells, and NRF2 negatively modulated apoptosis (FIG. 8E,F). Collectively, these results demonstrate that hyperglycemia reduces keratinocyte proliferation but increases apoptosis, and these effects can be attenuated by activation of the NRF2 pathway.

### Example X

Indeed, such experiments demonstrate a protective role for NRF2 against impaired diabetic wound healing was clearly demonstrated. First, perilesional tissues from diabetic and normoglycemic patients were analyzed and the results indicated that the diabetic wound skin tissues were under more severe oxidative stress than normal wound skin tissues, as demonstrated by greater oxidative DNA damage and activation of the NRF2 pathway. Next, Nrf2^{+/+} and Nrf2^{-/-} mice were used to demonstrate the importance of NRF2 in improving the impaired wound healing process in STZ-induced diabetes model. Diabetic Nrf2^{-/-} mice showed delayed wound closure when compared to diabetic Nrf2^{+/+} mice, which may be due to greater oxidative DNA damage, altered TGF-β1 and MMP9 expression, and increased apoptosis. Then, the therapeutic potential of NRF2 activators to restore normal wound healing was demonstrated in the Nrf2^{+/+} STZ-induced diabetic mouse model. In vitro experiments with HaCaT human keratinocytes further confirmed that NRF2 contributes to important events of wound healing including oxidative stress attenuation, promotion of proliferation and migration, and decreased apoptosis under high glucose incubation. These beneficial effects are, at least partially, through modulation of the expression of TGF-β1 and MMP9.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for treating a diabetes-related skin ulcer, comprising administering to a human patient suffering from a diabetes-related skin ulcer an effective amount of an NRF2 activating agent, wherein the NRF2 activating agent is able to activate the NRF2/KEAP1 pathway.

2. (canceled)

3. The method of claim 1, wherein the NRF2 activating agent is selected from cinnamaldehyde, sulforaphane, bordoxolone methyl, glutathione peroxidase-1 mimetic (e.g., ebselen), caffeic acid, resveratrol, curcumin, tanshinone I, tanshinone IIA, dihydrotanshinone, cryptotanshinone, and bixin.

4. The method of claim 1, wherein administration of the NRF2 activating agent results in one or more of the following:
reduces and/or prevents oxidative DNA damage within the diabetes-related skin ulcer;
increases TGF-β1 expression within the diabetes-related skin ulcer;
decreases MMP9 expression within the diabetes-related skin ulcer; and
reduces and/or prevents apoptosis within a diabetes-related skin ulcer.

5-7. (canceled)

8. The method of claim 1, wherein the human patient is one or more of the following:
suffering from diabetes mellitus; and
suffering from neuropathy.

9-11. (canceled)

12. The method of claim 1, wherein the diabetes-related ulcer is a chronic, non-healing skin ulcer.

13. The method of claim 1, wherein the diabetes-related ulcer is a foot ulcer.

14. The method of claim 13, wherein the foot ulcer is one or more of the following:
a neuropathy-related foot ulcer;
a trauma-related foot ulcer;
a deformity-related foot ulcer;
a high plantar pressure-related foot ulcer;
a callus formation-related foot ulcer;
an edema-related foot ulcer; and
a peripheral arterial disease-related foot ulcer.

15-20. (canceled)

21. The method of claim 1, wherein the NRF2 activating agent is administered topically, orally, and/or intravenously.

22. The method of claim 1, wherein the NRF2 activation agent is administered in a form selected from the group consisting of a cream form, a spray form, a dressing form, a patch form, a tablet form, and an intravenous form.

23. The method of claim 1, wherein the NRF2 activating agent is co-administered with one or more additional therapeutic agents effective for treating, ameliorating, and preventing diabetes-related skin ulcers.

24. The method of claim 23, wherein the one or more additional therapeutic agents are selected from a hemorrheologic agent (e.g., pentoxifylline, cilostazol), an antiplatelet agent (e.g., clopidogrel, aspirin), and a wound healing agent (e.g., becaplermin).

25. A method, comprising administering to a patient having a diabetes-related skin ulcer an NRF2 activating agent, wherein administration of the NRF2 activating agent results in one or more of the following:
a reduction and/or prevention of oxidative DNA damage within the diabetes-related skin ulcer;
an increase in TGF-β1 expression within the diabetes-related skin ulcer;
a decrease in MMP9 expression within the diabetes-related skin ulcer; and
reduction and/or prevention of apoptosis within the diabetes-related skin ulcer.

26-28. (canceled)

29. The method of claims 25-28, wherein the NRF2 activating agent is sulforaphane.

30. The method of claims 25-28, wherein the NRF2 activating agent is cinnamaldehyde.

31. The method of claim 25, wherein the human patient is one or more of a human being suffering from diabetes mellitus, a human patient is suffering from neuropathy, a human being at risk for developing a diabetes-related skin ulcer, and human being having a diabetes-related skin ulcer.

32. The method of claim 25, wherein the diabetes-related skin ulcer is a foot ulcer.

33. The method of claim 25, wherein the NRF2 activating agent is co-administered with one or more additional therapeutic agents effective for treating, ameliorating, and preventing diabetes-related skin ulcers, wherein the one or more additional therapeutic agents are selected from a hemorrheologic agent (e.g., pentoxifylline, cilostazol), an antiplatelet agent (e.g., clopidogrel, aspirin), and a wound healing agent (e.g., becaplermin).

34. (canceled)

35. The method of claim 25, wherein the NRF2 activating agent is administered topically, orally, and/or intravenously.

36. The method of claim 25, wherein the NRF2 activation agent is administered in a form selected from the group consisting of a cream form, a spray form, a dressing form, a patch form, a tablet form, and an intravenous form.

37. A kit comprising
one or more NRF2 activating agents (e.g., sulforaphane, cinnamaldehyde);
one or more additional therapeutic agents effective for treating, ameliorating, and preventing diabetes-related skin ulcers, wherein the one or more additional therapeutic agents are selected from a hemorrheologic agent (e.g., pentoxifylline, cilostazol), an antiplatelet agent (e.g., clopidogrel, aspirin), and a wound healing agent (e.g., becaplermin), and
instructions for administering the NRF2 agents to a subject.

38-39. (canceled)
